(19) United States
(12) Patent Application Publication
(10) Pub. No.: US 2016/0360721 A1
(43) Pub. Date: Dec. 15, 2016

(54) CANNABIS SATIVA PLANTS RICH IN CANNABICHROMENE AND ITS ACID, EXTRACTS THEREOF AND METHODS OF OBTAINING EXTRACTS THEREFROM

(71) Applicant: GW Pharma Limited, Salisbury (GB)

(72) Inventor: Etienne De Meijer, Salisbury (GB)

(73) Assignee: GW Pharma Limited, Salisbury (GB)

(21) Appl. No.: 15/153,839

(22) Filed: May 13, 2016

Related U.S. Application Data
(62) Division of application No. 12/936,947, filed on Dec. 22, 2010, now abandoned.

(30) Foreign Application Priority Data
Apr. 10, 2008 (GB) 0806553.4

(51) Int. Cl. A01H 5/12; A01G 1/00; A01H 1/02; A61K 31/352; A61K 36/185

(52) U.S. Cl. CPC A01H 5/12; A61K 31/352; A61K 36/185; A01H 1/02; A01G 1/001

(57) ABSTRACT

The present invention relates to plants producing, as their major cannabinoid cannabichromenic acid (CBCA) or its neutral (decarboxylated) form cannabichromene (CBC), hereafter jointly referred to as CBC(A). It additionally relates to; a botanical material obtainable from said plants; a botanical raw material (BRM), an extract including a botanical drug substance (BDS) and a purified BDS; a formulation comprising the BRM, BDS, purified BDS or other extract; the use of the BRM, BDS, purified BDS or other extract in the manufacture of a medicament; a method of deriving plants yielding a high proportion of the in cannabinoid CBC(A) at the expense of other cannabinoids; a method of cultivating plants such that they yield a high proportion of the cannabinoid CBC(A) at the expense of other cannabinoids; and a method of extracting CBC(A) from said plants.

### RELATED APPLICATIONS

This application is a divisional of U.S. application Ser. No. 12/936,947, filed on Dec. 22, 2010, which is a national stage filing under 35 U.S.C. §371 of international application PCT/GB2009/000947, filed Apr. 9, 2009, which was published under PCT Article 21(2) in English.

### INTRODUCTION

The present invention relates to plants producing, as their major cannabinoid cannabichromenic acid (CBCA) or its neutral (decarboxylated) form cannabichromene (CBC), hereafter jointly referred to as CBC(A).

It additionally relates to:
A botanical material obtainable from said plants;
A botanical raw material (BRM),
An extract including a botanical drug substance (BDS) and a purified BDS;
A formulation comprising the BRM, BDS, purified BDS or other extract;
The use of the BRM, BDS, purified BDS or other extract in the manufacture of a medicament;
A method of deriving plants yielding a high proportion of the cannabinoid CBC(A) at the expense of other cannabinoids;
A method of cultivating plants such that they yield a high proportion of the cannabinoid CBC(A) at the expense of other cannabinoids; and
A method of extracting CBC(A) from said plants.

### TECHNICAL FIELD

There are many different Cannabis sativa chemotypes. These comprise both undomesticated plants and cultivated varieties. The cultivated varieties include plants which have been cultivated as fibre producers (namely ones expressing low levels of the cannabinoid, tetrahydrocannabinol (THC) and it's acid (THCA) and high levels of the cannabinoid, cannabidiol (CBD) and it's acid (CBDA); those that have been bred (often illegally) for recreational use (i.e. ones expressing high THC and THCA levels) and more recently medicinal plants which have been selectively bred to express high levels of cannabinoids which are expressed at low levels in nature where typically the cannabinoids THC(A), CBD(A) and cannabinol (CBN) and its respective acid CBNA predominate.

Indeed, the present applicant has previously described breeding methods for obtaining plants rich in the cannabinoids THC, CBD and Cannabigerol (CBG). Meijer ERM de, Hammond K M (2005) The inheritance of chemical phenotype in Cannabis saliva L. (II): cannabigerol predominant plants. Euphytica 145: 189-198.

### BACKGROUND OF THE INVENTION

### Cannabinoid Biogenesis

The Cannabis plant synthesises and accumulates cannabinoids as carboxylic acids (e.g.. cannabichromenic acid, CBCA). In this specification the designation CBC(A) is used to refer to both the acid and its neutral form.

The most common cannabinoids are those with a pentyl side chain and include:
Cannabidiol (CBD);
Delta 9-tetrahydrocannabinol (THC);
Cannabichromene (CBC); and
Cannabigerol (CBG).

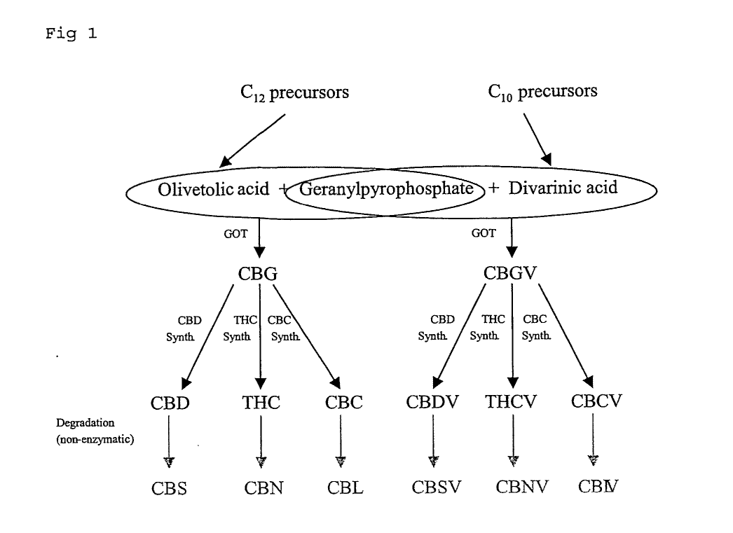

The first specific step in the pentyl cannabinoid biosynthesis is the condensation of geranylpyrophosphate (GPP) with olivetolic acid (OA) into CBG. (See FIG. 1). This reaction is catalysed by the enzyme geranylpyrophosphate:olivetolate geranyltransferase (GOT). CBG is the direct precursor for each of the compounds:
THC;
CBD; and
CBG.

The different conversions of CBG are enzymatically catalysed, and for each reaction an enzyme has been identified;
THC acid synthase;
CBD acid synthase: and
CBC acid synthase.

Similarly, cannabinoids with propyl side chains result if GPP condenses with divarinic acid (DA) instead of OA. The three cannabinoid synthase enzymes are not selective for the length of the alkyl side chain and convert cannabigerovarin (CBGV) into the propyl homologues of CBD, THC and CBC, which are indicated as;
Cannabidivarin (CBDV);
Delta 9-tetrahydrocannabivarin (THCV); and
Cannabichromevarin (CBCV), respectively.

Cannabinoids are deposited in the non-cellular, secretary cavity of glandular trichomes, Sirikantaramas et al 2005 (Plant. Cell Physiol 46: 1578-1582) confirmed the presence of the central precursor CBG and an exclusive THC synthase activity in the secretary cavity and concluded that this is not only the site of THC accumulation but also of its biosynthesis. As THC, CBD and CBC all result from CBG conversions, it was suggested that CBD and CBC are also synthesised in the secretary cavity.

Mahlberg and Kim (2004) (Journal of Industrial Hemp 9:15-36) reported that glands are exclusively specialised to synthesise high amounts of cannabinoids and that tissues other than glands contain only very low levels. They recognised three types of glandular trichomes:
A small bulbous form;
A large capitate-sessile form (both of which are present on leaf surfaces throughout the plant) and
A large capitate-stalked form that develops after flower initiation on inflorescence bracts (small leaves) and bracteoles (structures enclosing the ovary).

These authors report that the cannabinoid content of capitate-stalked glands is about 20 times that of capitate-sessile glands.

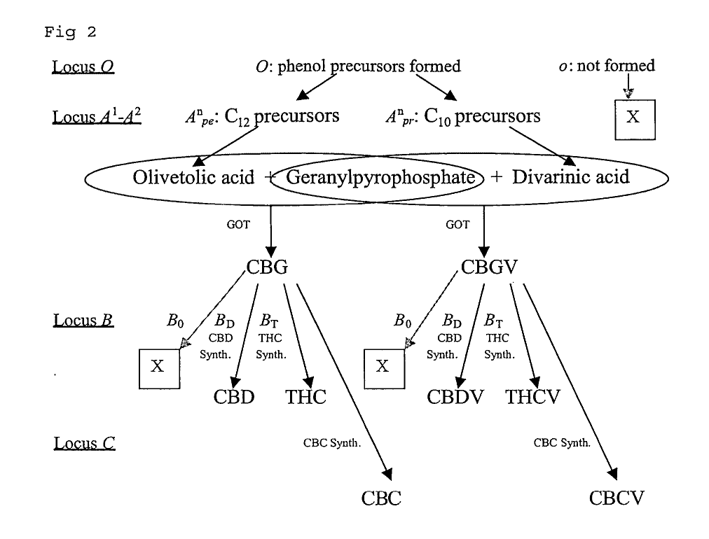

De Meijer et al. 2003 (Genetics 163: 335-346) previously concluded that the inheritance of CBD and THC composed chemotypes is controlled by a monogenic, co-dominant mechanism. A single locus referred to as B, with two alleles, B_{D} and B_{T}, encoding CBD and THC synthase respectively, was postulated, (See FIG. 2). According to this model, a true-breeding, strongly CBD predominant plant has a B_{D}/B_{D} genotype at the B locus, a true-breeding, strongly THC predominant plant has a B_{T}/B_{T} genotype and plants with substantial proportions of both CBD and THC are heterozygous B_{D}/B_{T}.

De Meijer and Hammond 2005 (Euphytica 145: 189-198) also concluded that plants accumulating the precursor CBG have a minimally functional mutation of B_{D}, called B_{D}, in the homozygous state, encoding for a weakened CBD synthase enzyme.

They had considered two possible options for CBC biosynthesis:
a further allele B_{D} at the B locus, encoding CBC synthase, or
the involvement of a completely different locus that may or may not be allelic.

There have been occasional reports of high CBC containing plants. Yotoriyama et al. 1980 (Yakugaku Zasshi 100: 611-614) presented a chromatogram of a Japanese THC-predominant fibre strain ‘Tochishi No. 1’, showing only a trace of CBD and a CBC peak with an area similar to the THC peak.

However, plants reaching substantial CBC proportions at maturity are uncommon,

Holley et al. 1975 (Journal of Pharmaceutical Sciences 64: 892-894) list samples from India with CBC proportions of up to 64% of the total cannabinoid fraction and with THC as the major complementary cannabinoid, although it must be stressed that the document does not specify whether these samples originated from mature plants. The applicant is inclined to disregard its relevance as it is most likely that the material examined was immature material.

For the reason that plants pure in CBC were not available, and the genetic control of CBC biosynthesis was unknown the applicant was not able to approach the development of high CBC containing plants in an obvious manner.

Accordingly, the applicant undertook a programme of research in order to try and establish a genetic model for CBC(A) chemotype inheritance in Cannabis. Thus, they set about exploring the mechanism that controls the CBC(A) proportion in the cannabinoid fraction. For that purpose breeding experiments were conducted with chemotypes characterised by contrasting CBC(A) proportions at maturity.

With the focus on CBC, a study of the ontogenetic variation in chemotype appeared necessary. This feature was examined by monitoring the cannabinoid composition of previously postulated genotypes and of chemotypes with relatively high CBG proportions yet a priori unknown underlying genotypes.

In the germplasm screening preceding the breeding programme, two accessions showing an unusual CBC proportion at maturity were identified:
A clone with a CBC proportion of 58%, and a complementary cannabinoid fraction dominated by CBD, was selected from an Afghan hashish landrace. RJ97.11. and
A Korean fibre landrace from Andong, which comprised mainly THC/CBC plants, with variable CBC proportions ranging from 7 to 58%. Two seedlings were selected 2000.577.118 and 2000.577.121.

These CBC rich breeding progenitors are shown in Table 1.

A plant bearing the genetic factors responsible for the two different prolonged juvenile phenotypes in the inbred lines RJ97.11 and 2000.577 has been deposited as seeds (NUMB 41541).

TABLE 1
Characteristics of materials used in the chemotype monitoring and the breeding experiments.

| | | | Putative | Cannabinoid composition ^{a} | | | | |
|---|---|---|---|---|---|---|---|---|
| Code | Generation/type | Source population | genotype | CBD | CBC | CBGM ^{b} | THC | CBG |

Lines used in chemotype monitoring experiment

| | | | | | | | | |
|---|---|---|---|---|---|---|---|---|
| 55.6.2.6.4.21 | S₄ inbred line | ‘Haze’, marijuana strain | B_{T}/B_{T} | 0.5 | 1.7 | 0.4 | 95.6 | 1.9 |
| 2001.22.6.20.14 | S₃ inbred line | (Afghan × Skunk) × | B_{D}/B_{D} | 91.2 | 2.9 | 1.0 | 3.7 | 1.2 |
| | | (Haze × Skunk) | | | | | | |
| 2002.2.4.42 | S₂ inbred line | (Afghan × Skunk) × S. | B₀/B₀ | 8.7 | 3.4 | 0.1 | 0.4 | 87.4 |
| | | Italian fibre hemp | | | | | | |
| RJ97.11.23 | S₂ inbred line | Afghan hashish | ? ^{c} | 61.9 | 30.6 | 4.2 | 2.5 | 0.8 |
| | | landrace | | | | | | |
| 2000.577.118.3.5 | S₃ inbred line | Korean fibre landrace | ? ^{c} | 0.8 | 22.4 | 7.3 | 69.3 | 0.2 |

CBC rich breeding progenitors

| | | | | | | | |
|---|---|---|---|---|---|---|---|
| 2000.577.118 | Non-inbred seedling | Korean fibre landrace | ? ^{c} | | 33.0 | 9.9 | 57.1 |
| 2000.577.121 | ″ | ″ | | | 39.5 | 7.8 | 52.7 |
| RJ97.11 | Non-inbred clone | Afghan hashish landrace | ? ^{c} | 33.2 | 57.8 | 6.8 | 2.2 |

^{a} The proportions (% w/w) of the individual cannabinoids in the total cannabinoid fraction assessed at maturity.
^{b} Cannabigerol-monomethylether.
^{c} A priori unknown.

### SUMMARY OF ME INVENTION

According to a first aspect of the present invention there is provided a Cannabis sativa plant producing as its major cannabinoid CBC(A), characterised in that it comprises at least one genetic factor encoding prolonged juvenile chemotype (PJC) and it has a B₀/B₀ genotype.

In one embodiment the at least one genetic factor encoding prolonged juvenile chemotype (PJC) is monogenic.

The monogenic factor may derive from an Afghan lineage (CBD(A) dory dominant chemotype) such as, for example, that designated RJ97.11.

In an alternative embodiment the at least one genetic factor encoding prolonged juvenile chemotype (PJC) is polygenic.

The polygenic factor may derive from a Korean lineage (THC(A) dominant chemotype) such as, for example, that designated 2000.577.118.

Indeed, the Cannabis sativa plant may comprise a plurality of genetic factors encoding prolonged juvenile chemotype (PJC).

The Cannabis sativa plant additionally comprising a B₀/B₀ genotype, such as that derived from Italian fibre hemp, isolate ISCI529/72 (also referred to as 2001/25) or more preferably, from a Ukranian fibre hemp, such as isolate USO 31. This cultivar is amongst several varieties of hemp that have been approved for commercial cultivation under subsection 39(1) of the Industrial Hemp Regulations in Canada for the year 2007.

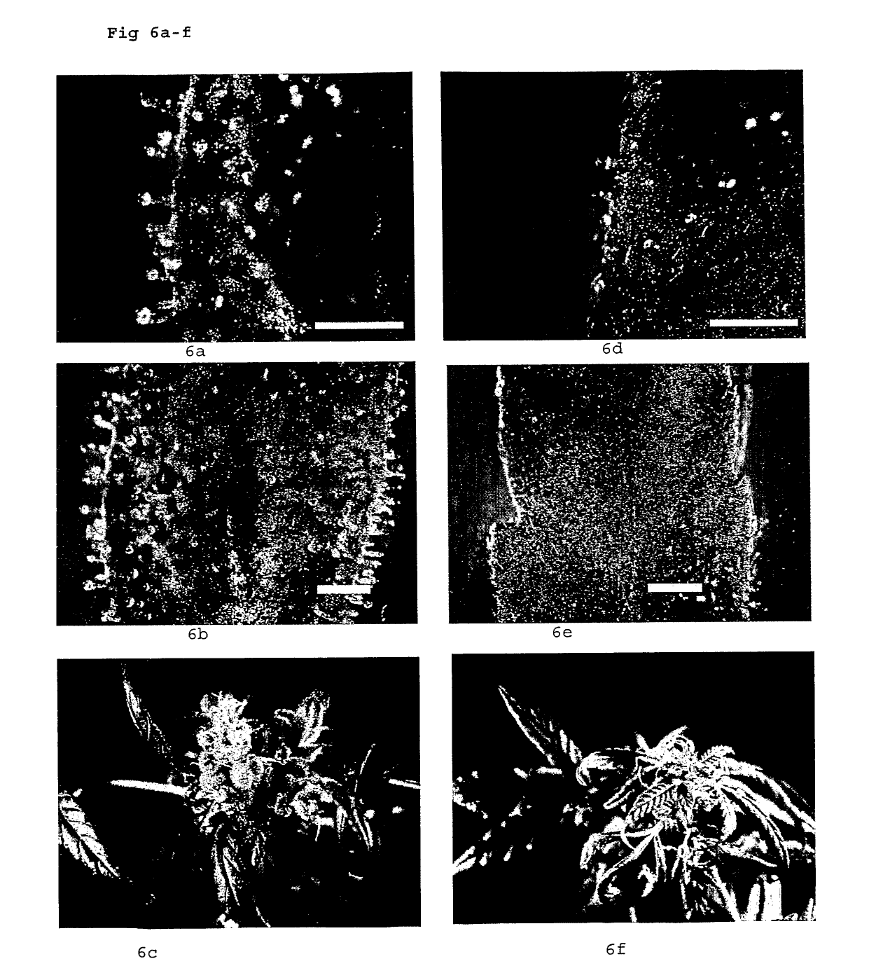

The Cannabis sativa plant phenotypically comprises leafy inflorescences with a few small bracteoles, and bracts that predominantly carried sessile glandular trichomes and substantially no stalked ones as illustrated in FIGS. 6d-f.

Preferably, the Cannabis sativa plant comprises, at maturity, greater than 65%, though 70%, 75%, 80%, 85%, 90% and 95% by weight CBC(A) based on the total weight of cannabinoids and may comprise as much as 98% or more by weight CBC(A) based on the total weight of cannabinoids.

Preferably, the Cannabis sativa plant comprises, at maturity, greater than 1% (w/w) of total cannabinoids in a Botanical Raw Material. More preferably still it comprises, at maturity, greater than 2% (w/w), more preferably still greater than 3% (wiw) of total cannabinoids in a Botanical Raw Material.

In yield terms the Cannabis sativa plant preferably provides a CBC(A) yield of greater than 5 g/m² from plant material grown to maturity, more preferably greater than 10 g/m² and most preferably a yield of greater than 15 g/m²from plant material grown to maturity. By maturity is meant the plant is subject to a thirteen week growth period.

According to a second aspect of the present invention there is provided a botanical material obtainable from the Cannabis sativa plants of the invention. The botanical material may be a botanical raw material (BRM) a botanical drug substance (BDS) or a purified BDS. The BDS may take the form of an extract which is preferably a standardised extract (standardised against characteristic markers). The terms used herein are those refered to in the Guidance for Industry Botanical drug products issued by the US department of health and human services (FDA) Centre for drug evaluation and research June 2004.

According to a third aspect of the present invention the BIDS or extract is formulated into a medicine. The formulation may include one or more excipients and the “active” extract may be formulated in a form suitable to its mode of administration which would include oral delivery, intravenous delivery, sub lingual delivery and all other forms standard in the pharmaceutical industry.

According to a fourth aspect of the present invention there is provided the use of the BDS or extract in the manufacture of a medicament for use in medicine.

The BDS or extract may be characterised by, for example:
HPLC;
LC or:
GC FID MS chromatography.

According to a fifth aspect of the present invention there is provided a method of deriving plants yielding a high proportion of the cannabinoid CBC(A) at the expense of other cannabinoids comprising:
a. Isolating/selecting a first plant comprising at least one genetic factor encoding prolonged juvenile chemotype (PJC);
b. Isolating/selecting a second plant comprising a B₀/B₀ genotype:
c. Crossing the first plant and second plant to obtain an F₁; and
d. Self-fertilising selected F₁ plants to obtain an F₂ generation and selecting those plants with a high proportion of the cannabinoid CBC(A) relative to other cannabinoids.

According to a sixth aspect of the present invention there is provided methodology for cultivating plants such that they yield a high proportion of the cannabinoid CBC(A) at the expense of other cannabinoids comprising:
a. Growing the plants under a defined reduced light intensity, and/ or
b. A defined reduced generative phase.

Light intensity can be defined by the level of photosynthetically active radiation PAR measured in W/m² or cumulative PAR measured in MJ/m². A reduced light intensity (for growing cannabis plants) would be less than 17.45 MJ/m² or 67.4 W/m²

Typically a cannabis plant which is grown from cuttings is subject to 5 weeks (35 days) of vegetative growth (usually under 24 h light) and then 8 weeks (56 days) of generative growth (usually under 12 h light). Total 13 weeks (91 days).

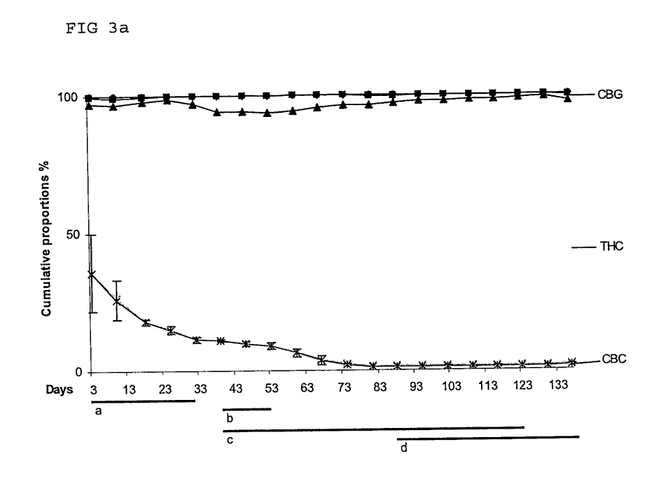

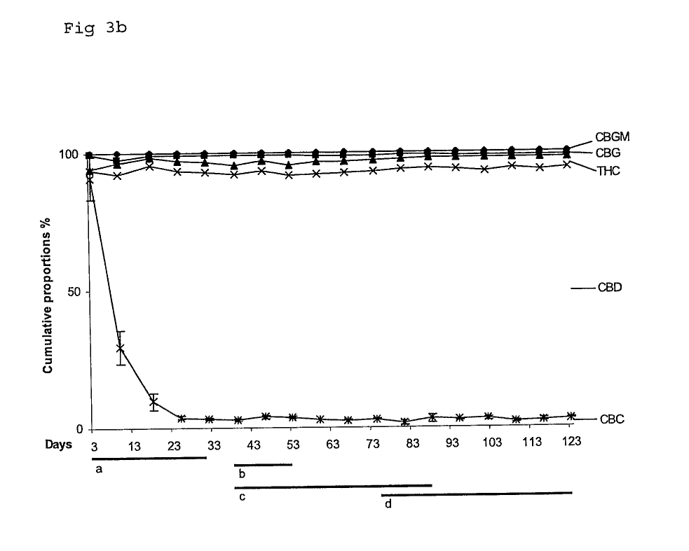

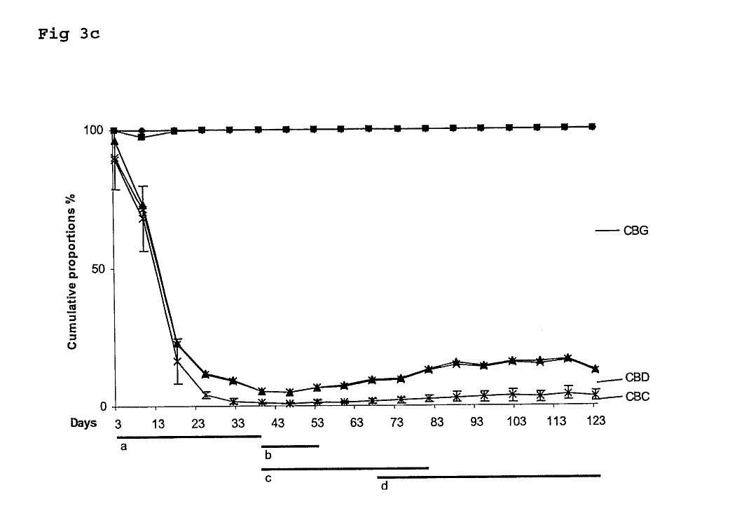

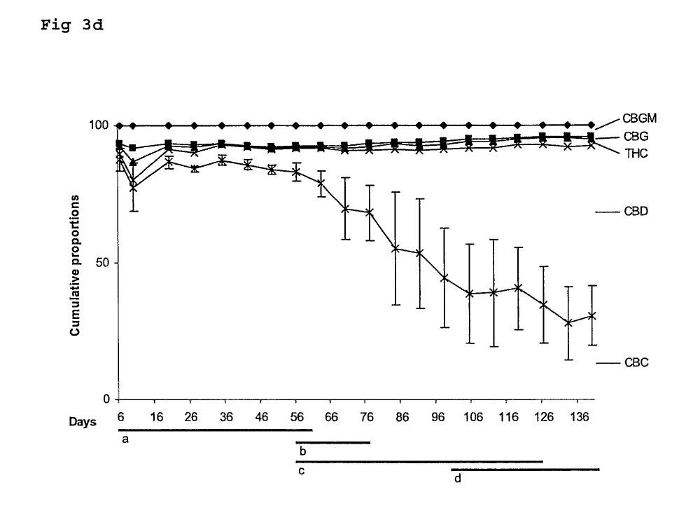

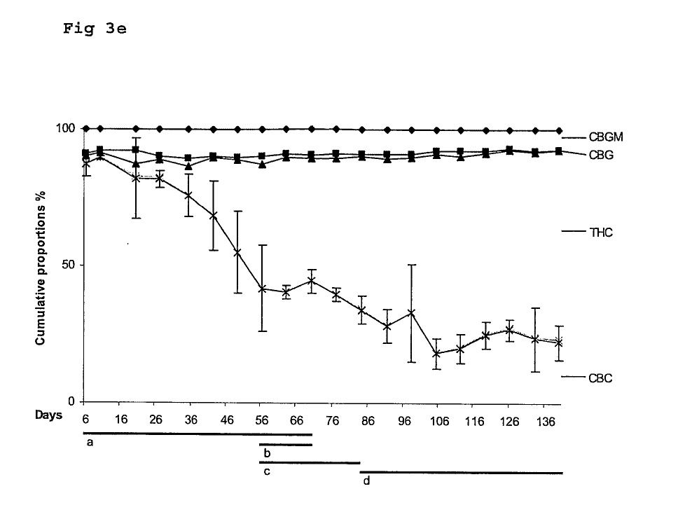

A reduced generative phase is thus one of less than 8 weeks (from day 35), and may be measured in days or weeks. Preferably the reduced generative phase is less than 7 weeks, more preferably less than 6 weeks, more preferably still less than 5 weeks and most preferably about 4 weeks in length (See e.g. FIG. 3d.)

According to a seventh aspect of the present invention there is provided a method of extracting CBC(A) from Cannabis plant material comprising selectively separating trichomes from plant material and then selecting sessile trichomes.

The separation of trichomes into sessile trichomes and stalked trichomes may be done based on their size.

Surprisingly it has been found that these different trichomes contain differing contents of different cannabinoids and sessile trichomes have been found to be a source of highly pure CBC(A).

Agitating fresh cannabis material in e.g. icy water and then sieving the suspension through respectively a 73 μm sieve and a 25 μm sieve separates the glandular (larger) trichomes from the sessile (smaller) ones.

According to an eighth aspect of the present invention there is provided a plant extract (BDS) comprising at least 64% by weight CBC(A) relative to the total cannabinoid content of the extract.

The invention will be further described, by way of example only, with reference to the in following figures and examples in which:

FIG. 1 is a diagramatic representation of the cannabinoid biosynthesis pathways;

FIG. 2 is a diagramatic representation of the cannabinoid biosynthesis together with its (postulated) genetic control;

FIGS. 3a-3e: Illustrate the cannabinoid composition, represented as cumulative proportions of the total cannabinoid fraction, in the course of the life time of various inbred lines in which.

FIG. 3a: is a true-breeding THC predominant inbred line (putative genotype B_{T}/B_{T});

FIG. 3b: is a true-breeding CBD predominant inbred line (putative genotype B_{D}/B_{D});

FIG. 3c: is a true-breeding CBG predominant inbred line (putative genotype B₀/B₀);

FIG. 3d: is an inbred line directly derived from the Afghan RJ97.11 source clone; and

FIG. 3e: is an inbred line directly derived from the Korean 2000.577.118 seedling.

In each the X-axes represents the sampling time in days from seedling emergence.

Solid lines under the X-axes specify the tissue that was sampled:
(a) is the latest expanded apical stem leaves;
(b) is the latest expanded inflorescence leaves;
(c) is bracteoles, bracts and leaves from inflorescences with white, immature stigma; and
(d) is bracteoles, bracts and leaves from inflorescences with brown, mature stigma.

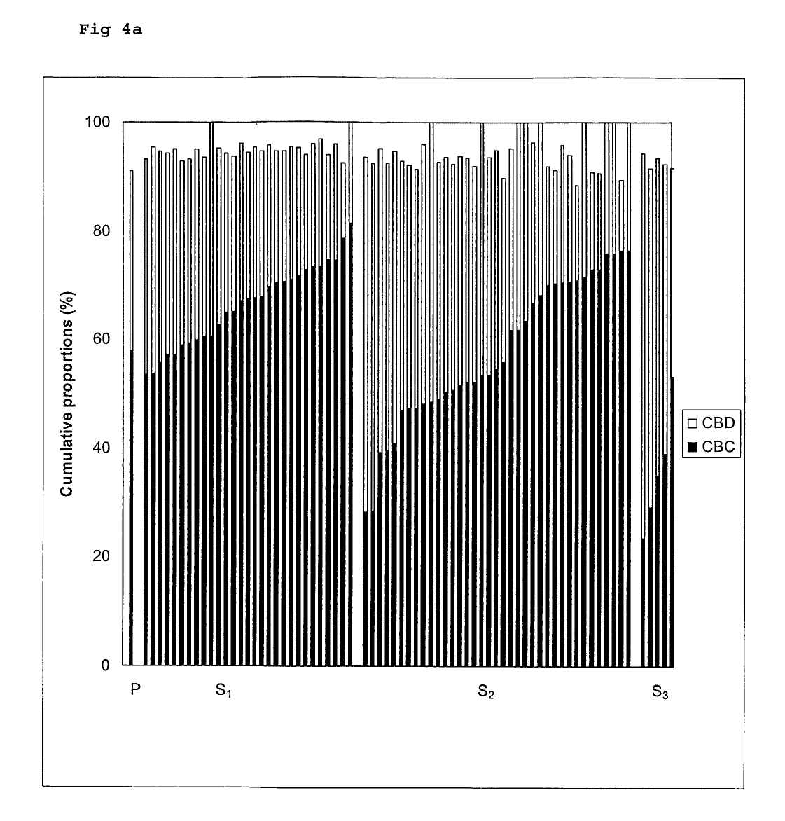

FIGS. 4a-4b: are stack bar diagrams showing the cannabinoid composition of:

FIG. 4a: parental clone RJ97.11 and its S₁, S₂ and S₃ inbred offspring; and

FIG. 4b: parental seedlings 2000.577.118 and 0.121 and their S₁, S₂ and S₃ inbred offspring.

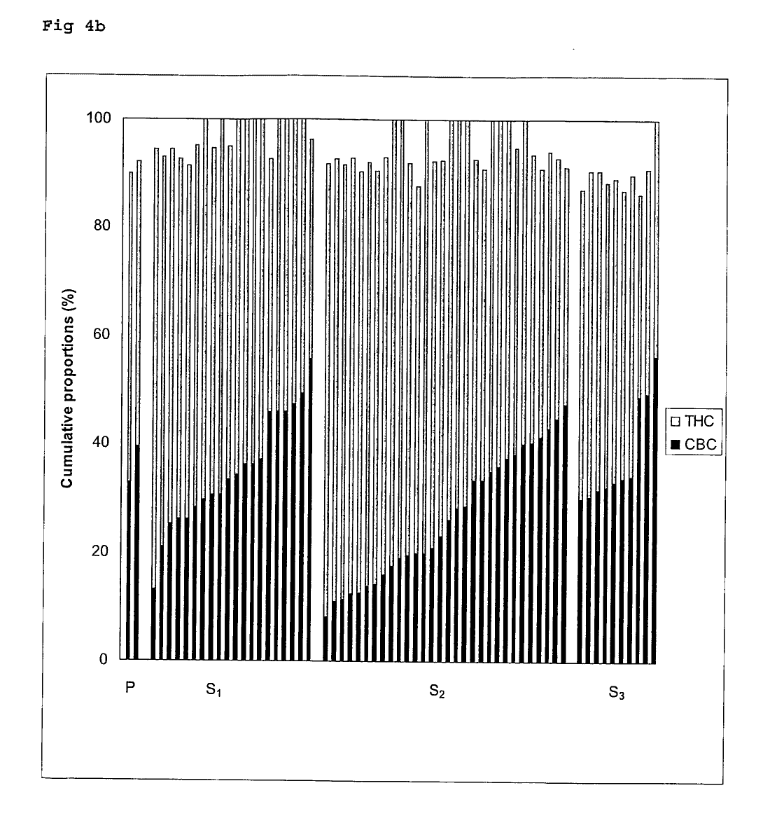

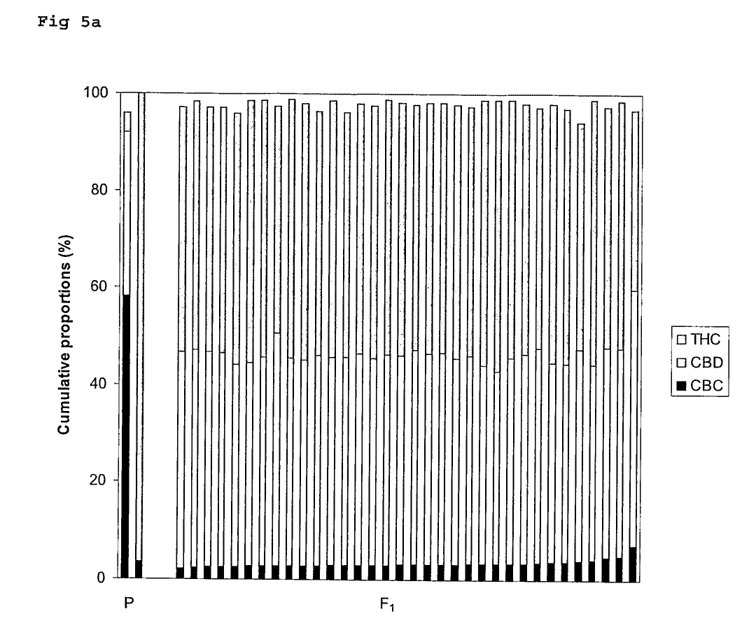

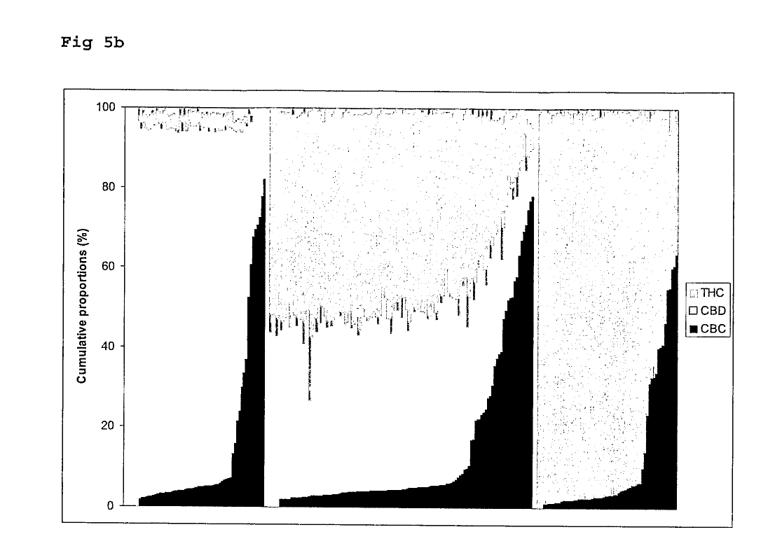

FIGS. 5a-5b: are stack bar diagrams showing the cannabinoid composition of the clone RJ97.11 and a true-breeding THC predominant plant.

FIG. 5a is their hybrid F_{1;} and

FIG. 5b is their hybrid F₂.

For the representation of the F₂, the 244 plants were primarily classified on the basis of their CBD/THC content ratio. Subsequently, within the three resulting groups, individuals were sorted by increasing proportion of CBC.

FIGS. 6a-6f are photographs of mature floral tissue of different F₂ segregants:

FIGS. 6a-6c are of a wild type segregant with negligible CBC(A) in which:

FIG. 6a shows bract surface detail (bar 100 μm);

FIG. 6b shows bract surface overview (bar 5 mm); and

FIG. 6c shows the entire flower cluster.

FIGS. 6d-6f are of a PJC segregant relatively rich CBC(A) in which:

FIG. 6d shows bract surface detail (bar 100 μm)

FIG. 6e shows bract surface overview (bar 5 mm)

FIG. 6f shows the entire flower cluster.

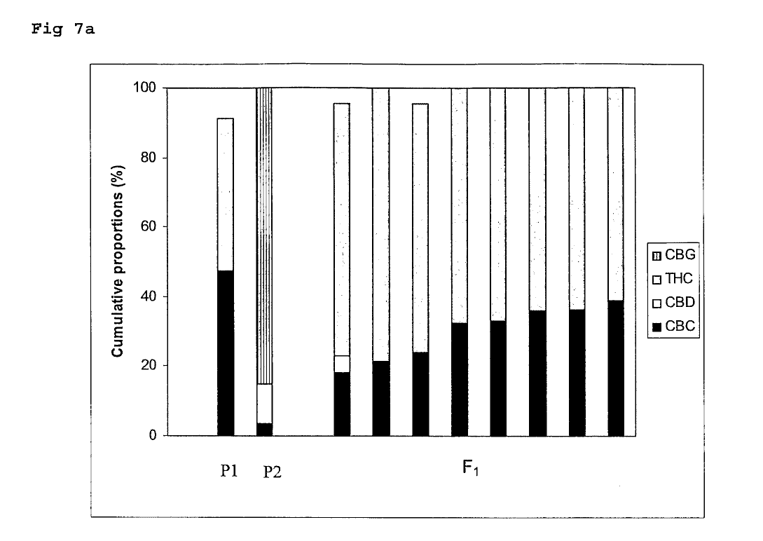

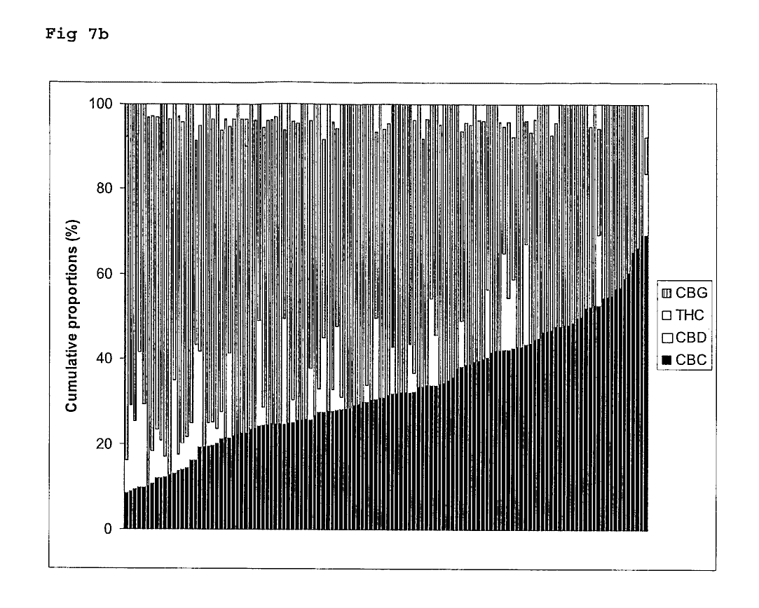

FIGS. 7a-7b: are stack bar diagrams showing the cannabinoid composition of the inbred seedling 2000.577.188.3.7 (P1) and a true-breeding CBG predominant plant (P2)

FIG. 7a is their hybrid F_{1;} and

FIG. 7b is their hybrid F₂.

For both generations, plants were sorted by increasing proportion of CBC.

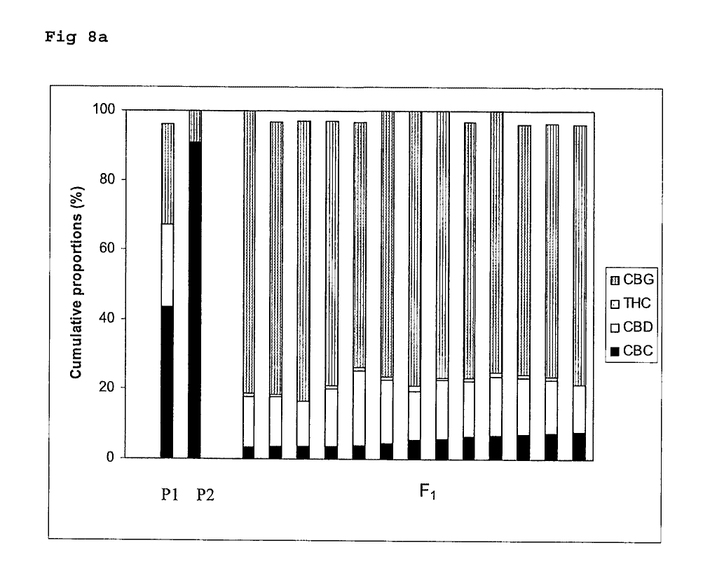

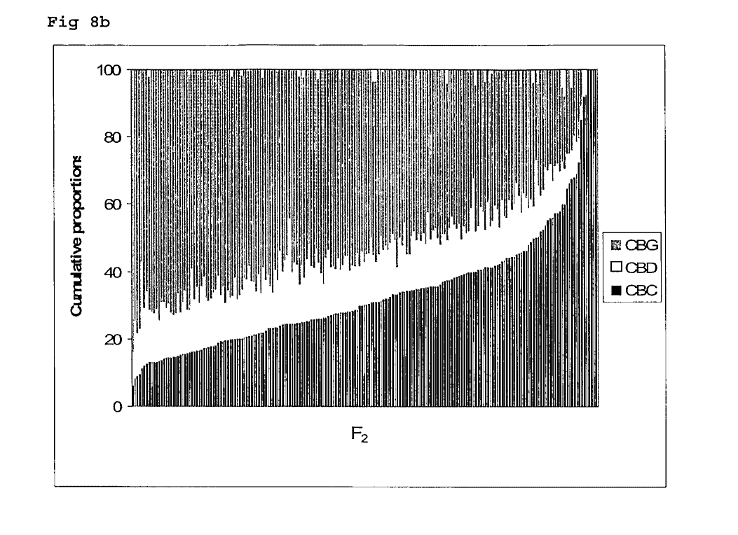

FIGS. 8a-8b: are stack bar diagram showing cannabinoid composition.

FIG. 8a is of a high P_{CBC} inbred offspring individual P1 selected from a (Korean×CBG predominant) progeny, a high P_{CBC} inbred clone P2 originating from an (Afghan×CBG predominant) progeny and their hybrid F_{1;} and

FIG. 8b is the F2 obtained from a self fertilised F1 plant; and

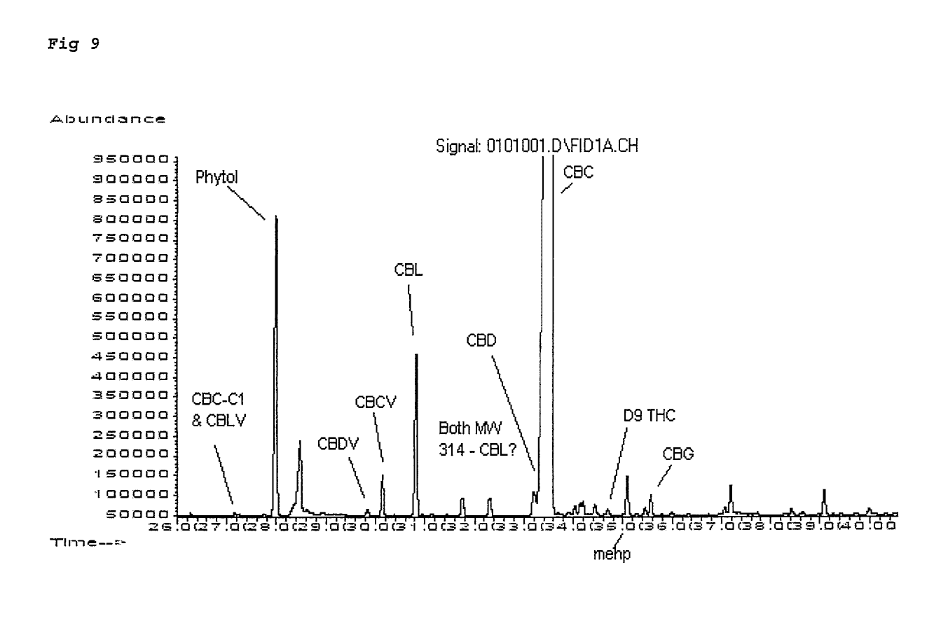

FIG. 9 is a GC-FID-MS chromatogram of a CBC(A) plant of the invention.

### DETAILED DESCRIPTION

The experiments described below were undertaken in order to determine CBC(A) inheritance in Cannabis sativa. For that purpose breeding experiments were conducted with chemotypes characterised by contrasting CBC proportions at maturity. With the focus on CBC(A), a study of the ontogenetically and environmentally (light intensity) induced variation in chemotype also appeared appropriate.

From the results, the applicant has been able to breed plants with a novel CBC(A) rich chemotype, and obtain therefrom botanical raw materials (BRM), and novel extracts which can be used in medicine.

### EXAMPLE 1

### Chemotype Monitoring Experiment

### 1.1 Materials and Methods

Table 1 presents five female inbred lines that were grown for periodic assessments of their cannabinoid contents. Up to five seedlings per line were evaluated under similar glasshouse conditions.

Plants were kept under permanent light for the first two weeks after emergence. Then, to induce flowering, the 24 h photoperiod was dropped to 19 h and further gradually reduced by 15 minutes per day. When the photoperiod reached the level of 11 h, it was kept constant until the end of the experiment. The onset of flowering was visible in all plants by the day the 11 h photoperiod was reached. Commencing shortly after seedling emergence, at weekly intervals, and always around mid-day, samples were taken from the most recently developed tissues. These were, in order:
(a) The last expanded apical stem leaves;
(b) The last expanded inflorescence leaves;
(c) Bracteoles, bracts and leaves from inflorescences with white, immature stigma; and
(d) Bracteoles, bracts and leaves from inflorescences with brown, mature stigma.

In addition, the question of whether the same tissue shows changes in cannabinoid composition in the course of ageing was investigated. For this purpose leaflets were periodically sampled from a fixed leaf pair at the 3^{rd} or 4^{th} stem, node (sample type ‘e’).

Per plant, per sampling date, the samples were individually extracted and analysed as described below. The respective cannabinoid contents were totalised and the individual cannabinoids were quantified as relative proportions of the total content. Per accession, per sampling date, mean cannabinoid proportions were calculated.

### 1.2 Results

FIGS. 3a-e present the cannabinoid composition during the life cycle, as assessed in the latest developed tissues, of:
True-breeding THC predominant (FIG. 3a);
CBD predominant (FIG. 3b);
CBG predominant (FIG. 3c); and
Afghan (FIG. 3d) and Korean inbred lines (FIG. 3e) (Both High CBC).

All the lines considered showed a strong presence of CBC shortly after emergence which declined with ageing. The plants predominant in THC at maturity had a CBC proportion in the total cannabinoid fraction (P_{CBC}) of about 40% three days after emergence. This proportion gradually decreased over a 10-week period and stabilised at about 1-3% in the immature floral samples (FIG. 3a).

The first true leaves of the lines predominant in CBD and CBG at maturity had a P_{CBC} of about 90%. Then the P_{CBC} rapidly reduced and after only three weeks, still in the stage that primary stem leaves were sampled, a level of about 1-5% was reached. This percentage remained stable for their remaining lifetime (FIGS. 3b and 3c).

The Afghan and Korean inbred lines showed a P_{CBC} of about 90% shortly after emergence which decreased more slowly than in the aforementioned materials and stabilised at the more substantial level of about 25% of the cannabinoid fraction of the mature floral samples (FIGS. 3d and 3e).

The true-breeding THC, CBD and CBG predominant inbred lines showed an increase in total cannabinoid content during the sampling period from about 0.8-11%, 0.7-10% and 0.25-4%, respectively. This parameter was therefore negatively correlated with the declining P_{CBC} value (r=−0.80, −0.38 and −0.57 for the three lines respectively).

In the Afghan and the Korean inbred lines, the total cannabinoid content varied between 1-3% throughout the sampling period and showed little correlation with P_{CBC}.

The ‘e’ samples which were periodically taken from different leaflets of a fixed primary leaf pair, preserved the same cannabinoid composition throughout the entire sampling period in all the accessions (data not shown).

### EXAMPLE 2

### Breeding Experiments

### 2.1 Method

The CBC rich breeding progenitors selected for the experiments were:
A female clone RJ97.11 obtained from HortaPharm B.V., Amsterdam, The Netherlands; and
A Korean fibre landrace 2000.577, from the Cannabis collection at. Plant Research International (formerly CPRO), Wageningen, The Netherlands,

From the later two female seedlings are identified by the suffix:
0.118; and
0.121.

All progenies were produced from female parents only. In order to self-fertilise or mutually cross female plants, a partial masculinisation was chemically induced. Isolating plants in paper bags throughout the generative stage ensured the self-fertilisations.

The distribution of chemotypes in segregating progenies was determined and X² values were calculated to test the conformity of observed segregation ratios to those expected on the basis of hypothesised models. Three sets of breeding experiments were performed:
2.1.1 Direct inbreeding of the source materials with a high CBC proportion;
2.1.2 Crossing of material with a high CBC proportion (original source material or inbred offspring directly derived from it) with either:
   2.1.2.1 various THC predominant materials (putative genotype B_{T}/B_{T}. de Meijer et al. 2003); and
   2.1.2.2 various CBG predominant materials (putative genotype B₀/B₀, de Meijer and Hammond 2005)
(and inbreeding of the resulting progenies); and
2.1.3 Mutual crossing of two different high CBC inbred lines, one based on the Afghan and the other on the Korean parental source and inbreeding the resulting progeny.

### 2.2 Results

2.2.1 Inbreeding of Progenitors with a High Proportion of CBC

In the RJ97.11 parental plant and in its entire inbred offspring, the proportion of [CBC+CBD] on average accounted for 94.6% of the total cannabinoid fraction. The remaining fraction consisted almost entirely of cannabigerol-monomethyl-ether (CBGM).

A few individuals also had a trace of THC.

Within the inbred generations of RJ97.11, the absolute contents of CBC and CBD were uncorrelated: r=0.17, 0.06 and −0.11 for the S₁, S₂ and S₃ respectively.

Table 2 gives means and standard deviations for the total cannabinoid content and P_{CBC} of the successive inbred generations from RJ97.11. In the course of inbreeding there was no systematic trend noticeable in either the mean values or the variabilities of these characteristics.

TABLE 2
The total cannabinoid content and the proportion of CBC
in the cannabinoid fraction in the successive inbred
generations from the source materials RJ97.11 and 2000.577.

| | | | Total cannabinoid | Proportion |
|---|---|---|---|---|
| Source | | No. of | content (% w/w) | of CBC (%) |
| accession | Generation | plants | Mean ± Std. | Mean ± Std. |
| RJ97.11 | S₀ | 1 | 3.88 | 57.8 |
| | S₁ | 29 | 2.93 ± 0.72 | 66.3 ± 7.4 |
| | S₂ | 37 | 2.69 ± 0.84 | 57.7 ± 13.4 |
| | S₃ | 5 | 3.48 ± 0.65 | 36.0 ± 10.1 |
| 2000.577 | S₀ | 2 | 1.47 ± 0.22 | 36.2 ± 3.3 |
| | S₁ | 20 | 1.34 ± 0.54 | 35.0 ± 10.5 |
| | S₂ | 30 | 3.71 ± 1.87 | 26.5 ± 11.7 |
| | S₃ | 10 | 2.67 ± 0.97 | 38.0 ± 9.2 |

The cannabinoid profile of the RJ97.11 parental plant and the inbred generations are visualised in the stack bar diagram of FIG. 4a. The S₁ is based on the single RJ97.11 parent. The fertility in this material declined sharply with the level of inbreeding so in order to evaluate a reasonable number of individuals, the S₂s and S₃s in FIG. 4a include the inbred progeny from several plants of the previous generation. Within generations, the variation in the cannabinoid proportions was considerable, but discontinuity in the pattern of cannabinoid' composition was not observed and the parental plant and the consecutive inbred generations of this line were essentially constant in respect of their CBC/CBD chemotype.

In the two parental plants from the 2000.577 population that were inbred and in their offspring, CBC and THC together occupied on average 94.7% of the cannabinoid fraction, with CBGM being the single additional cannabinoid. The absolute contents of CBC and THC within the inbred generations of 2000.577 showed limited correlation: r=0.12, 0.21 and 0.66 for the S₁, S₂ and S₃, respectively. Means and standard deviations for the total cannabinoid content and P_{CBC} of the 2000.577 inbred generations did not show a systematic trend (Table 2). Within generations, the variation in the cannabinoid proportions was substantial but gradual and there was no segregation into discrete chemotypes. The parental mixed CBC/THC chemotype was expressed by all individuals of the generations observed in (FIG. 4b).

2.2.2 Crosses of Afghan High P_{CBC} Plants with Various THC and CBG Predominant Materials

The total proportion of [CBC+CBD+THC+CBG] in all the parental plants considered and in their hybrid offspring, occupied at least 89.3 and on average 98.9% of the total cannabinoid fraction. The remaining fraction consisted solely of CBGM. All 14 F₁s, irrespective of whether they resulted from crosses of Afghan derived plants with true breeding THC predominant or CBG predominant plants, were chemotypically uniform and only had a limited P_{CBC} (Table 3).

TABLE 3
Chemotypical data for F₁ and F₂ progenies resulting from crosses between Afghan plants (P1) with a high proportion of CBC (P_{CBC}),
and various true breeding THC and CBG predominant materials (P2).

| | | | | | | P_{CBC} | No. of | P_{CBC} | No. of | | | |
|---|---|---|---|---|---|---|---|---|---|---|---|---|
| | | P_{CBC} values | | | No. | ranges | plants, F₂ | ranges | plants, F₂ | | 3:1 | R-value ^{c} |
| | F₂ | | | F₁ | of F₁ | F₂ low ^{a} | low P_{CBC} | F₂ high ^{a} | high P_{CBC} | X² | accepted | Ctot-P_{CBC} |
| Cross | Progeny | P1 | P2 | min-avg-max | plants | (min-max) | group | (min-max) | group | value ^{b} | p = 0.05 | (F₂s) |
| 1 | A | 71.4 | 1.8 | 3.2-4.2-7.7 | 32 | 0.0-5.3 | 5 | 38.9-69.5 | 4 | 1.81 | Yes | −0.70 |
| | B | | | | | 0.0-14.0 | 39 | 27.5-73.6 | 9 | 1.00 | Yes | −0.51 |
| 2 | C | 77.5 | 1.8 | 1.9-3.1-8.2 | 24 | 1.3-9.0 | 35 | 30.0-60.6 | 12 | 0.01 | Yes | −0.66 |
| 3 | D | 77.5 | 1.5 | 3.1-3.5-4.0 | 2 | 0.0-12.9 | 78 | 18.2-91.8 | 23 | 0.27 | Yes | −0.62 |
| 4 | E | 63.9 | 1.5 | 4.0-5.0-5.9 | 2 | 0.9-6.8 | 10 | 25.2-84.5 | 6 | 1.33 | Yes | −0.66 |
| 5 | F | 71.4 | 2.5 | 3.3-4.3-5.3 | 9 | 0.0-7.8 | 29 | 15.1-58.7 | 13 | 0.79 | Yes | −0.70 |
| | G | | | | | 1.3-7.4 | 39 | 17.9-69.1 | 6 | 3.27 | Yes | −0.66 |
| 6 | H | 71.4 | 0.3 | -7.1- | 1 | 0.0-8.6 | 19 | 55.0-90.9 | 3 | 1.52 | Yes | −0.48 |
| 7 | I | 71.4 | 1.5 | -8.9- | 1 | 2.7-12.1 | 27 | 14.5-95.4 | 10 | 0.08 | Yes | −0.48 |
| 8 | J | 77.5 | 0.0 | 2.2-5.2-7.9 | 7 | 0.0-11.1 | 57 | 14.7-94.6 | 18 | 0.04 | Yes | −0.32 |
| 9 | K | 63.9 | 2.5 | 2.7-2.9-3.3 | 4 | 0.0-5.8 | 38 | 22.6-37.4 | 5 | 4.10 | No | −0.60 |
| 10 | L | 63.9 | 2.2 | 4.9-7.1-10.8 | 21 | 0.0-10.9 | 47 | 14.1-87.0 | 31 | 9.04 | No | −0.61 |
| 11 | M | 58.3 | 3.5 | 2.2-3.3-7.2 | 34 | 0.0-10.0 | 40 | 14.1-71.1 | 12 | 0.10 | Yes | −0.48 |
| | N | | | | | 0.0-13.3 | 77 | 17.0-78.3 | 20 | 0.99 | Yes | −0.64 |
| | O | | | | | 0.0-10.2 | 69 | 17.4-82.2 | 26 | 0.28 | Yes | −0.59 |
| 12 | P | 41.4 | 0.0 | 0.0-2.2-10.1 | 47 | 0.0-13.6 | 71 | 17.9-100.0 | 25 | 0.06 | Yes | −0.20 |
| 13 | Q | 39.0 | 0.0 | 0.0-1.0-3.6 | 22 | 0.0-13.0 | 52 | 17.6-100.0 | 25 | 2.29 | Yes | −0.48 |
| 14 | R | 57.8 | 2.9 | 2.3-4.8-12.3 | 28 | 0.0-12.6 | 13 | 23.1-36.7 | 2 | 1.09 | Yes | −0.28 |
| | S | | | | | 0.0-4.9 | 10 | 23.2-34.9 | 2 | 0.44 | Yes | −0.41 |
| | All | | | | | | 755 | | 252 | 0.00 | Yes | |

^{a} Per F₂ the segregant groups ‘low P_{CBC}’ and ‘high _{PCBC}’ were discriminated on the basis of a discontinuity in the range of sorted P_{CBC} values.
^{b} X² values were calculated to test conformity to the model of a single Mendelian locus with a recessive allele, encoding ‘high P_{CBC}’ and a dominant allele encoding ‘low P_{CBC}’. The X² threshold for acceptance at p = 0.05 is 3.84.
^{c} The coefficient of correlation between the total cannabinoid content and P_{CBC}.

Hybrids resulting from an Afghan×THC predominant cross had chemotypes predominated by CBD and THC and within an F₁ the absolute CBD and THC contents were strongly correlated (r values generally 0.8-0.9).

All F₁ plants resulting from Afghan×CBG predominant crosses were strongly CBD predominant.

The stack bar diagram of FIG. 5a presents the chemotypes of the parental plants and the F₁s of one of the Afghan×THC predominant crosses (Table 3, cross no, 11). FIG. 5b shows the distribution of chemotypes in the large pooled F₂ (Table 3, F₂s M, N and O) that was based on three randomly chosen inbred F₁ plants from this cross and comprised 244 individuals. Irrespective of the CBC proportion. 59 plants with a THC/CBD content ratio ranging from 0.00 to 0.053 were CBD predominant: 121 contained both THC and CBD in a comparable proportion (THC/CBD content ratio range 0.33-3.88) and 64 plants were THC predominant (THC/CBD content ratio range 18.87-∞). With a X² value of 0.22, a 1:2:1 segregation ratio is readily accepted (threshold for acceptance at p=0.05: X²<5.99). Within the three discrete segregant groups based on the THC/CBD content ratios, individuals in FIG. 5b are sorted by increasing P_{CBC}. It appears that within each group, the first three quarters of the plants have low P_{CBC} values up to approximately 8% whereas, after a sudden increase, the latter quarter shows P_{CBC} values of 15-80%. A higher P_{CBC} was observed in individuals with relatively low total cannabinoid content. For the 244 F₂ plants presented in FIG. 5b, these two characteristics were negatively correlated (r=−0.51).

Chemotypical data on P_{CBC} for all the 14 crosses between Afghan high P_{CBC} plants and various low P_{CBC}, THC or CBG predominant materials is summarised in Table 3. In all the F₂s, comparable distributions of the P_{CBC} values were found as illustrated in FIG. 5b, and there was also a consistent negative correlation between P_{CBC} and the total cannabinoid content. When ranked by P_{CBC} value, all F₂ progenies showed a clear discontinuity in the P_{CBC} inclination trend. It separates ca. 75% of the individuals with a narrow range of lower values from ca. 25% with a wide range of higher values. A P_{CBC} value of 14% can be considered as a general threshold value to demarcate these two groups, Individuals with P_{CBC}≦14% belong to the low P_{CBC} group, those with P_{CBC}>14% to the high P_{CBC} group. For 17 of the 19 F₂s that were considered, X² tests accepted a 3:1 segregation ratio for the low P_{CBC} versus the high P_{CBC} group.

All F₂s from the Afghan×THC predominant crosses segregated into fairly pure CBD plants, mixed CBD/THC plants and fairly pure THC plants in a 1:2:1 ratio (accepted by X² tests, data not shown), based on discontinuities in the THC/CBD ratio of the complementary cannabinoid fraction and irrespective of P_{CBC}. The segregation was clear-cut. General THC/CBD value ranges for the chemotype classes over all F₂s of this type were: CBD predominant (0≦THC/CBD≦0.09), mixed THC/CBD (0.26≦THC/CBD≦3.88) and THC predominant (11.71≦THC/CBD≦∞).

Data on the dihybrid segregation of the characters, P_{CBC} value and THC/CBD ratio are summarised in Table 4a. For all F₂s, X² tests accepted a 3:6:3:1:21 segregation ratio for the variants (low P_{CBC}-CBD predominant): (low P_{CBC}-mixed THC/CBD): (low P_{CBC}-THC predominant): (high P_{CBC}-CBD predominant): (high P_{CBC}-mixed THC/CBD): (high P_{CBC}-THC predominant).

TABLE 4a
Dihybrid segregation in F₂ progenies resulting from crosses between Afghan high
P_{CBC} plants, with a complementary fraction of mainly CBD, and various low P_{CBC}
true breeding THC predominant materials. Per progeny, per chemotype category,
the number of individuals is given.

| | P_{CBC} low | | | P_{CBC} high | | | | | 3:6:3:1:2:1 |
|---|---|---|---|---|---|---|---|---|---|
| | | CBD/ | | | CBD/ | | | | accepted |
| Progeny | CBD | THC | THC | CBD | THC | THC | Total | X^{2 a} | p = 0.05 |
| A | 3 | 2 | 0 | 1 | 1 | 2 | 9 | 7.30 | Yes |
| B | 10 | 19 | 10 | 2 | 7 | 0 | 48 | 3.78 | Yes |
| C | 8 | 12 | 15 | 2 | 6 | 4 | 47 | 6.90 | Yes |
| F | 10 | 10 | 9 | 3 | 7 | 3 | 42 | 3.52 | Yes |
| G | 14 | 15 | 10 | 3 | 2 | 1 | 45 | 7.68 | Yes |
| K | 10 | 15 | 13 | 1 | 3 | 1 | 43 | 6.74 | Yes |
| M | 8 | 22 | 10 | 2 | 5 | 5 | 52 | 2.41 | Yes |
| N | 21 | 37 | 19 | 2 | 13 | 5 | 97 | 3.45 | Yes |
| O | 16 | 33 | 20 | 10 | 11 | 5 | 95 | 3.64 | Yes |
| R | 5 | 3 | 5 | 0 | 2 | 0 | 15 | 6.51 | Yes |
| S | 3 | 4 | 3 | 0 | 2 | 0 | 12 | 2.22 | Yes |
| All | 108 | 172 | 114 | 26 | 59 | 26 | 505 | 9.64 | Yes |

^{a} X² values were calculated to test conformity to the model of two independent Mendelian loci.

According to this model one locus has a recessive allele, encoding ‘high P_{CBC}’, and a dominant allele encoding ‘low P_{CBC}’. The other locus has two codominant alleles, encoding either CBD or THC predominance when homozygous, and a mixed CBD/THC chemotype when heterozygous. The X² threshold for acceptance at p=0.05 is 11.07.

Based on the predominance of either CBG or CBD in the cannabinoid fraction complementary to CBC, the F₂s from the Afghan×CBG predominant crosses segregated consistently into CBD predominant versus CBG predominant plants in a 3:1 ratio (accepted by X² tests, data not shown). Five plants could not be classified by this criterion (Table 4b, footnote^{b}).

TABLE 4b
Dihybrid segregation in F₂ progenies resulting from crosses between Afghan high
P_{CBC} plants, with a complementary fraction of mainly CBD and various low P_{CBC},
true breeding CBG predominant materials. Per progeny, per chemotype category,
the number of individuals is given.

| | | | P_{CBC} high | | | | |
|---|---|---|---|---|---|---|---|
| | | | Cannabinoid | Cannabinoid | | | |
| | P_{CBC} low | | complement | complement | | | 9:3:3:1 |
| | CBD | CBG | CBD | CBG | | | accepted |
| Progeny | predominant | predominant | preodominant | predominant | Total | X^{2 a} | p = 0.05 |
| D | 62 | 16 | 13 | 10 | 101 | 4.95 | Yes |
| E | 7 | 3 | 5 | 1 | 16 | 1.78 | Yes |
| H | 16 | 3 | 3 | 0 | 22 | 3.05 | Yes |
| I | 18 | 9 | 7 | 3 | 37 | 1.20 | Yes |
| J | 43 | 14 | 12 | 6 | 75 | 0.69 | Yes |
| L | 43 | 4 | 20 | 11 | 78 | 17.41 | No |
| P ^{b} | 57 | 14 | 16 | 8 | 95 | 1.95 | Yes |
| Q ^{b} | 43 | 9 | 15 | 6 | 73 | 2.28 | Yes |
| All | 289 | 72 | 91 | 45 | 497 | 11.44 | No |

^{a} X² values were calculated to test conformity to the model of two independent Mendelian loci. According to this model one locus has a recessive allele, encoding ‘high P_{CBC}’ and a dominant allele encoding ‘low P_{CBC}’. The other locus has a recessive allele, encoding CBG predominance and a dominant allele encoding CBD predominance. The X² threshold for acceptance at p = 0.05 is 7.82.
^{b} From the progenies P and Q, one and four high P_{CBC} individuals, respectively, were excluded.

In these plants CBC was the single cannabinoid detected and they could not be further classified on the basis of a complementary cannabinoid fraction.

Data on the dihybrid segregation of the characters, P_{CBC}-value and predominance of either CBD or CBG in the complementary cannabinoid fraction, are summarised in Table 4b. For seven of the eight F₂s, X² tests accepted a 9:33:1 ratio for the variants (low P_{CBC}-CBD predominant): (low P_{CBC}-CBG predominant): (high P_{CBC}-CBD predominant): (high P_{CBC}-CBG predominant).

As with the Afghan high P_{CBC} progenitor, the high P_{CBC} segregants did not produce the usual resinous flower clusters. Instead, they had leafy inflorescences with a few small bracteoles, and bracts that only carried sessile glandular trichomes and no stalked ones (FIG. 6d-f).

2.1.2.2 Cross of Korean High P_{CBC} Material with CBG Predominant Material

The total proportion of [CBC+CBD+THC+CBG] in the parental plants and in their hybrid offspring, occupied at least 91.3 and on average 98.0% of the total cannabinoid fraction. The remaining fraction was purely CBGM.

The F₁ resulting from the cross of the Korean inbred (S₃) line 2000.577.118.3.7 and a true breeding CBG predominant inbred line was uniform for chemotype (FIG. 7a). With a value range of 18.1-39.0%, P_{CBC} was much higher than in the F₁s obtained with Afghan plants. The average P_{CBC} value of the eight F₁ plants was 30.0%, which is close to the parental average P_{CBC} value of 25.5%. Besides CBC, THC was the major cannabinoid in all F₁ plants and some individuals also had a minor proportion of CBD and/or CBGM. The F₁ individuals were self-fertilised to produce inbred F₂s. The chemotypes of the pooled F₂ plants, sorted by P_{CBC} are presented in FIG. 7b. The F₂ achieved a much wider P_{CBC} range than the F₁; 8.6-69.3%. The average P_{CBC} of the 122 F₂ plants was 33.1%. In contrast with the F₂s obtained with Afghan plants, the pattern of P_{CBC} values did not show any discontinuity and the distribution of individuals over P_{CBC} classes followed a Gaussian pattern.

In alignment with the F₂s obtained with Afghan progenitors, P_{CBC} in this F₂ was also negatively correlated with the total cannabinoid content (r=−0.58). All Korean based high P_{CBC} plants had a poor plant habit in respect of drug production. The inflorescences were very open, floral bracts were virtually absent and the bracteoles were small and poorly covered with stalked glandular trichomes.

In the F₂, CBC was accompanied by a complementary cannabinoid fraction predominated by either THC (in 90 plants) or CBG (in 32 plants). With a X² value of 0.10. a 3:1 segregation ratio for THC-versus GBG predominant is readily accepted (threshold for acceptance at p=0.05: X²<3.84).

### 2.2.3 Mutual Cross of Afghan Based and Korean Based High P_{CBC} Inbred Material

A high P_{CBC} inbred individual selected from the (Korean×CBG predominant) progeny was crossed with a selected high P_{CBC} inbred clone originating from an (Afghan×CBG predominant) progeny. The total proportion of [CBC+CBD+THC+CBG] in the parental and offspring plants occupied on average 97.9% of the total cannabinoid fraction with CBGM being the single complementary cannabinoid. FIG. 8a presents the chemotypes of the parents and the F₁. The CBC proportion of the F₁ individuals is greatly reduced in comparison with the parental plants. The minimal, average and maximal P_{CBC} levels in the F₁ were 3.1-5.3-7.7%. The average total cannabinoid content of the 13 F₁ plants was 9.2% (range 7.4-11.2%) which by far exceeds the parental total cannabinoid contents of ca. 1% (Korean based parent) and ca. 4% (Afghan based parent). Besides CBC, the complementary cannabinoid fraction of the F₁s was consistently CBG predominant with a residual presence of CBD. In contrast with the parents, the F₁ individuals had fairly dense floral clusters consisting of bracteoles and bracts that were covered with normal densities of stalked glandular trichomes. A large F₂ generation of 195 individuals, obtained from a single F₁ plant, was evaluated. The total cannabinoid content ranged from 0.83 to 10.99% and P_{CBC} ranged from 6.23 to 100%, and both parameters were negatively correlated (r=−0.46). The ranked P_{CBC} values showed a slow trend for the majority of the P_{CBC} values and a somewhat steeper inclination for a minority at the end (FIG. 8b). F₂ individuals with high CBC proportions showed the phenotypical features as illustrated for such plants in FIG. 6d-f. As in the F₁, the complementary cannabinoid fraction was consistently CBG predominant with a residual presence of CBD. Some F₂ plants contained a minor proportion of the CBC degradant cannabicyclol (CBL).

### EXAMPLE 3

### CBC(A) Content and Vegetative State

### 3.1 Methodology

In order to determine whether a certain presence of CBC is a universal, albeit transitory, characteristic of Cannabis, early stem leaves from 178 vegetative cuttings from a variety of source populations, were analysed for cannabinoid content.

### 3.2 Results

The early vegetative leaves from all accessions contained CBC. It was the major cannabinoid in 4.5% of the samples and the second in 78%.

### EXAMPLE 4

### Effect of Light Intensity

### 4.1 Methodology

It was noticed that plants tended to show higher CBC proportions when, for self-fertilisation, they were grown in paper isolation bags. To investigate this effect systematically, five CBC rich female clones were grown under different levels of photosynthetically active radiation (PAR).

Two clones (M240, M271) were derived from the Afghan breeding source, one (M274) from the Korean breeding source, and two (M272, M273) were selected from Afghan B₀/B₀×Korean B₀/B₀ cross progenies.

In M271, the cannabinoid fraction complementary to CBC, was a mixture of CBD and THC in comparable amounts. In the other clones the complementary cannabinoid fraction was dominated by CBG.

Initially, all cuttings were kept under identical conditions of permanent light: a two week rooting phase under an average PAR level of 7/57 W/m² (12/12 h) and, after transplanting to 3 litre pots, another two weeks of vegetative development under an average PAR level of 38/94 W/m² (12/12 h). (i.e. 4 week vegetative growth.)

For generative development and maturation, they were then subjected to a 12 h photoperiod for 60 days. During this stage the cuttings were placed under different levels of PAR, an average of 67.4, 37.9, 23.3 and 0.9 W/m² respectively, measured just above the canopy. The four areas with varying light levels were constructed in a single glasshouse compartment with horizontal and vertical shading of different densities. Fans were installed for sufficient air circulation. Temperature and relative air humidity did not differ between the four light levels. Per regime, five to eight cuttings per done were fully randomised and spaced at a density of 16 plants /m². Edging plants of similar age and size were used to avoid margin effects on the test cuttings. PAR values were automatically recorded at five-minute intervals and for the entire generative stage cumulative PAR was approximated in MJ/m² per light regime.

At maturity, the botanical raw material of each cutting (BRM: the total mass of leaves, floral leaves, bracts and bracteoles) was dried, weighed and homogenised and its cannabinoid content and cannabinoid composition were assessed. Yields of BRM and cannabinoids in g/cutting were multiplied by 16 to obtain yields in g/m². Per clone, treatment effects were tested (Anova F-test, p=0.05) and treatment means were compared pair-wise by Fisher's LSD method (p=0.05).

### 4.2 Results

Five CBC rich clones were grown under different light intensities during a 60 days generative period. (Eight and a half weeks) Cumulative PAR values for the four light regimes were estimated at 17.45, 9.82, 6.03 and 0.23 MJ/m², respectively.

Under the most reduced light level, all plants died within the first two weeks of the experiment. Under the remaining regimes, variable numbers of plants survived until the end of the experiment. Their physiological maturity was demonstrated by a limited seed set due to a slight monoeciousness in one of the clones. Results for these regimes are presented in Table 5.

With a reduction of light, all five clones showed an upward trend in P_{CBC}. Those from the 6.03 MJ/m² area had a significantly (p=0.05) higher P_{CBC} value than those under 17.45 MJ/m². Mutually, the clones differed considerably in the height and width of their achieved P_{CBC} range on the full 0-100% scale. No significant effect of light level on the absolute CBC content was found in the dry botanical raw material of four of the clones. Only the CBC content of M271 was significantly affected, but in this case light levels and CBC contents did not show a coherent trend. In contrast, with reduced light, the total cannabinoid content decreased significantly in four clones. With the exception of clone M274, the resultant CBC yield dropped considerably with reducing light, mainly due to a decreasing yield of botanical dry matter.

TABLE 5
Means for the yield of dry botanical raw material (BRM), the total cannabinoid- (Ctot) and CBC content in the
homogenised BRM, the proportion of CBC in the total cannabinoid fraction (P_{CBC}) and the resulting CBC yield,
for five clones grown under three different light levels during a 60 days generative period. Light levels are indicated
by the cumulative PAR estimated for the entire generative period. Means are based on the cuttings that survived
until the end of the experiment. Per column, per clone, means showing a common letter are not different at p = 0.05.

| | Cumulative | No. of | No. of | | | | | CBC^{a} |
|---|---|---|---|---|---|---|---|---|
| | PAR | cuttings | surviving | BRM, yield | Ctot | CBC content^{a} | P_{CBC}^{a} | yield |
| Clone | (MJ/m²) | tested | cuttings | (g/m²) | (% w/w) | (% w/w) | (% w/w) | (g/m²) |
| M240 | 17.45 | 7 | 7 | 356 a | 2.46 a | 1.18 a | 49.0 a | 4.27 a |
| | 9.82 | 7 | 7 | 174 b | 2.55 a | 1.40 a | 56.3 a | 2.50 b |
| | 6.03 | 7 | 4 | 144 b | 1.50 b | 1.21 a | 82.5 b | 1.76 b |
| M271 | 17.45 | 8 | 8 | 821 a | 2.75 a | 1.88 b | 6.82 a | 15.46 a |
| | 9.82 | 8 | 8 | 483 b | 2.98 a | 2.10 a | 70.3 ab | 10.20 b |
| | 6.03 | 8 | 8 | 248 c | 2.14 b | 1.53 c | 71.6 b | 3.87 c |
| M272 | 17.45 | 5 | 5 | 258 a | 2.04 a | 1.85 a | 90.7 a | 4.79 a |
| | 9.82 | 5 | 5 | 120 b | 1.92 a | 1.85 a | 96.7 b | 2.18 b |
| | 6.03 | 5 | 1 | 53 b | 1.61 a | 1.61 a | 100.0 b | 0.85 b |
| M273 | 17.45 | 6 | 6 | 257 a | 3.61 a | 0.53 a | 14.7 a | 1.35 a |
| | 9.82 | 6 | 5 | 172 ab | 2.58 b | 0.59 a | 24.0 b | 0.95 ab |
| | 6.03 | 6 | 2 | 109 b | 1.36 c | 0.45 a | 33.5 b | 0.49 b |
| M274 | 17.45 | 6 | 5 | 203 a | 1.28 a | 0.45 a | 35.5 a | 0.91 a |
| | 9.82 | 6 | 5 | 126 a | 0.92 b | 0.39 a | 42.8 b | 0.48 a |
| | 6.03 | 6 | 2 | 226 a | 0.69 b | 0.30 a | 43.8 b | 0.68 a |

^{a}‘CBC’ refers to the in totaled detected CBC alkyl homologues and degradants (CBC, CBCV, CBL)

### EXAMPLE 5

### The Assessment of Cannabinoid Composition in Proximal and Distal Parts of Floral Bracts

### 5.1 Methodology

The possibility that CBC synthase activity is restricted to sessile glandular trichomes was considered as an explanation for the trends in cannabinoid composition observed during plant development. Floral bracts where glandular stalked trichomes were only apparent in the proximal region, close to the petiole, were selected. These bracts also carry sessile trichomes that are fairly evenly distributed over the entire surface so are suitable material to detect possible metabolic differences between sessile and stalked trichomes. The proximal and distal parts of these floral bracts from clones with THC-, CBD- and CBG predominant chemotypes were sampled separately and analysed for their cannabinoid content. Sessile and stalked glandular trichomes on the bract parts were counted using a light microscope at a magnification factor of 40. Per clone, for the distal as well as for the proximal parts of the bract, 20 areas of 16.5 mm² each were examined on the upper- and 20 on the lower surfaces, and the mean densities of sessile and stalked trichomes on the distal and proximal parts were calculated.

### 5.2 Results

Three CBD, three THC and two CBG predominant clones were used for a comparison between the proximal and distal parts of their floral bracts, focusing on the densities of glandular trichomes and the cannabinoid composition. Similar results were found for the different clones.

The density of glandular stalked trichomes in the proximal area was 100× that of the distal parts (3 per mm² vs. 0.03 per mm²).

Mean densities of sessile trichomes on proximal and distal parts were of the same order of magnitude (0.44 and 0.29 per mm², respectively).

The CBC content in proximal and distal parts was 0.05 and 0.04% vew, respectively, but the total cannabinoid contents were higher in the proximal than in the distal parts (1.90 and 0.68% w/w, respectively). The proportion of CBC in the total cannabinoid fraction (P_{CBC}) was somewhat lower in the cannabinoid-rich proximal parts than in the distal parts (3.34 and 5.56% w/w, respectively).

### EXAMPLE 6

### BDS Analysis

### 6.1 Results

A GC-FID-MS chromatogram of the BDS obtained from one clone M240 is illustrated in FIG. 9. It shows a major CBC peak at around 34 min with a series of lesser peaks, some of which are identified. On analysis, Table 6, the CBC content of the cannabinoids was found to be 89.9%.

TABLE 6

| Cannabinoid | Content % pa |
|---|---|
| CBC | 89.9 |
| CBL | 4.4 |
| CBCV | 1.0 |
| CBD | 1.0 |
| CBL2? | 0.8 |
| CBL3? | 0.7 |
| CBG | 0.5 |
| CBDV | 0.2 |
| THC | 0.1 |
| CBC-C1 | 0.1 |

### EXAMPLE 7

### Trichome Separation Methodology and Comparison of the Cannabinold Content of Sessile and Glandular Trichomes

This series of tests evaluated a method of removing trichomes from cannabis material described by Jansen and Terris 2002 (Journal of Cannabis Therapeutics 2002: 2(3-4):135-143). The published work described the efficient collection of glandular trichome. However, it did not state if the sessile, as well as glandular stalked, trichomes were removed. In these tests, trichomes were removed. Sessile and large stalked glandular trichomes were separated using appropriate size filters.

Fresh or dried cannabis material was thoroughly mixed with slurry of ice and water, using a domestic food mixer. As manual judgement of texture confirmed, the glandular trichome heads hardened at low temperatures and were readily separated from the trichome stalk cells during mixing. Being heavier than water the resin heads sank, and were then separated from the pulp by pouring the mixture though a fine sieves (220 μm approximate mesh). The resin heads passed through and the ‘spent pulp’ was retained. The resin heads were then efficiently separated from the bulk of the water by pouring the suspension through a 73 μm sieve and then a finer 25 μm sieve.

In theory, the resin heads from glandular stalked trichomes (reported typical diameter 75-199 μm) should have been trapped on the 73 μm sieve, whilst the sessile trichomes (typically 50 μm) fall through and are caught on the 25 μm mesh.

The resin heads collected from h sieve were moved and frozen or dried prior to further study or use, as is appropriate.

The preparation of sessile trichome glandular trichome heads collected from vegetative leaves of clone M240 was dried overnight. The resin proved an extremely potent and pure source of CBC. The CBC potency was 44% w/w and it constituted 94% of the cannabinoid total (Table 7).

TABLE 7

| | |
|---|---|
| | % |
| % w/w | Purity |

| Cannabinoid | CBC | CBCV | CBG | CBD | CBC |
|---|---|---|---|---|---|
| Mean | 44.37 | 0.57 | 0.33 | 0.60 | 94.18 |
| sd | 4.95 | 0.07 | 0.04 | 0.06 | 0.25 |

### CONCLUSIONS FROM THE EXAMPLES

Whilst in nature there can be found Cannabis sativa plants which exhibit a prolonged juvenile chemotype (PJC) the one or more genetic factors responsible for this prolonged expression are expressed together with a range of other cannabinoids leaving mixed cannabinoid extracts.

As a consequence of identifying and understanding the genetic lad for these PJC plants the applicant has been able to cross these plants with plants having a B₀/B₀ genotype to selectively breed plants which are highly selective for CBC(A). The utility of such plants in the pharmaceutical industry is readily apparent.

Additionally, by growing the plants under defined conditions e.g. reduced light intensity and/or for a shortened period, extracts with a higher purity of CBC content can be obtained (albeit at a reduced yield).

Furthermore, the use of techniques which are selective for e.g. sessile trichomes can additionally be used to improve selectivity in extracts.

A likely explanation for the benefits derived from the PJC containing Afghan and Korean plants is that in contrast to wild-type plants, where the CBC synthase gene may only be expressed in the juvenile state these plants have an inheritable factor, which causes gene expression of CBG synthase to be maintained throughout the adult stage.

The crosses between plants with contrasting CBC proportions demonstrated that the genetic factor responsible for PJC has a monogenic, recessive nature as far as the Afghan lineage (based on RJ97.11) is concerned. The dihybrid segregation data indicates that this factor is inherited independently from locus B.

A contrasting P_{CBC} cross with the involvement of a Korean high P_{CBC} parent yielded an F₁ with a gradual range of intermediate P_{CBC} values and an F₂ that did not segregate for P_{CBC}. This suggests a different, polygenic background for PJC in the Korean material.

1. A Cannabis sativa plant producing as its major cannabinoid cannabichromenic acid or cannabichromene (CBC(A)), characterised in that it comprises at least one genetic factor encoding prolonged juvenile chemotype (PJC) and it has a B₀/B₀ genotype, wherein the at least one genetic factor encoding prolonged juvenile chemotype (PJC) is monogenic.

2. (canceled)

3. The Cannabis sativa plant as claimed in claim 1 wherein the monogenic factor is derived from RJ97.11.

4.-7. (canceled)

8. The Cannabis sativa plant as claimed in claim 1 wherein the B₀/B₀ genotype is derived from ISCI529/72 or from USO 31.

9. (canceled)

10. The Cannabis sativa plant as claimed in claim 1 characterised in that morphologically it comprises leafy inflorescences with a few small bracteoles, and bracts that predominantly carry sessile glandular trichomes and substantially no stalked ones as illustrated in FIGS. 6d-f.

11. The Cannabis sativa plant as claimed in claim 1 comprising, at maturity, greater than 65% by weight CBC(A) based on the total weight of cannabinoids.

12. The Cannabis sativa plant as claimed in claim 11 which comprises greater than 98% by weight CBC(A) based on the total weight of cannabinoids.

13. The Cannabis sativa plant as claimed in claim 11 comprising, at maturity, greater than 1% (w/w) of total cannabinoids in a Botanical Raw Material.

14. The Cannabis sativa plant as claimed in claim 13 comprising, at maturity, greater than 3% (w/w) of total cannabinoids in a Botanical Raw Material.

15. The Cannabis sativa plant as claimed in claim 1 providing a CBC(A) yield of greater than 5 g/m² from plant material grown to maturity under 12 h/day light during the generative growth phase.

16. The Cannabis sativa plant as claimed in claim 15 providing a yield of greater than 15 g/m² from plant material grown to maturity under 12 h/day light during the generative growth phase.

17. (canceled)

18. A botanical material, botanical raw material (BRM), botanical drug substance (BDS), purified BDS or an extract obtainable from a Cannabis sativa plant as claimed in claim 1.

19. A formulation comprising BDS, purified BDS or other extract obtainable from a Cannabis sativa plant as claimed in claim 1 and one or more excipients.

20. A BDS, purified BDS or extract obtainable from a Cannabis sativa plant as claimed in claim 1 for use in medicine.

21. The BDS as claimed in claim 20 characterised in that it has a CBC GC-FID-MS chromatotographic fingerprint substantially as illustrated in FIG. 9 with a major CBC peak at around 34 min and a plurality of lesser minor cannabinoid peaks.

22. The BDS as claimed in claim 21 wherein the CBC comprises at least 85% of the cannabinoid content.

23. A method of deriving plants yielding a high proportion of the cannabinoid CBC(A) at the expense of other cannabinoids comprising
a. Isolating/selecting a first plant comprising at least one genetic factor encoding prolonged juvenile chemotype (PJC);
b. Isolating/selecting a second plant comprising a Bo/Bo genotype;
c. Crossing the first plant and second plant to obtain an F1; and
d. Self-fertilising selected F1 plants to obtain an F2 generation and selecting those plants with a high proportion of the cannabinoid CBC(A) relative to other cannabinoids.

24. Methodology for cultivating plants such that they yield a high proportion of the cannabinoid CBC(A) at the expense of other cannabinoids comprising:
a) Growing the plants under a defined reduced light intensity, and/ or
b) A defined reduced generative phase.

25. The method as claimed in claim 24 wherein the light intensity is less than a cumulative photosynthetically active radiation (PAR) of 17.45 MJ/m2.

26. The method as claimed in claim 24 wherein the generative phase is less than 8 weeks.

27. The botanical drug substance (BDS) as claimed in claim 20 comprising at least 64% by weight CBC(A) by weight relative to the total cannabinoid content.
